(19) United States
(12) Patent Application Publication
(10) Pub. No.: US 2006/0084880 A1
(43) Pub. Date: Apr. 20, 2006

(54) System for bioelectrical interaction with an individual with a simplified electrode fixation

(75) Inventors: Olaf Such, AACHEN (DE); Josef Lauter, Geilenkirchen (DE); Harald Reiter, Aachen (DE); Ralf Schmidt, Aachen (DE); Andras Montvay, Stuttgart (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 10/534,479

(22) PCT Filed: Nov. 5, 2003
(86) PCT No.: PCT/IB03/04969

§ 371 Date: May 10, 2005

(30) Foreign Application Priority Data
Nov. 15, 2002 (EP) 02079761.9

(51) Int. Cl. A61B 5/0402

(52) U.S. Cl. 600/509

(57) ABSTRACT

A wearable system (1) arranged to enable a bioelectrical interaction with an individual when being brought into a contact with the individual's skin (S). The system (1) comprises an electronic device with electrodes (3) to enable said interaction. The electrodes (3) are shaped to enable the fixation of the device on a suitable carrier (7), said carrier being provided with receiving portions (5) to enable said fixation.

A system for bioelectrical interaction with an individual with a simplified electrode fixation The invention relates to a wearable system arranged for enabling a bioelectrical interaction with an individual when being brought into contact with the individual's skin, said system comprising an electronic device arranged to be mounted on a carrier, said electronic device comprising electrodes arranged to carry out said interaction.

A system as set forth in the opening paragraph is known from DE 20112175. The known system comprises a sensor arrangement provided with electrodes to enable an electrical interaction with the individual. In particular, the known system is arranged to measure electrical signals propagating in the body of the individual as a result of a physiological activity. To enable a fixation of the known system to the body of the individual, the sensor arrangement is provided with a separate fixation arrangement in order to enable the fixation of the sensor arrangement to the carrier, which in its turn is attached to the body of the individual.

A disadvantage of the known system is a necessity for provision of a separate fixation arrangement to enable the fixation of the sensor arrangement to the carrier. Moreover, the fixation arrangement has to meet special requirements for purposes of enabling a durable interaction of the system with the individual. For example, on one hand, an external pressure applied to the electrodes must satisfy the conditions of being sufficiently high to immobilize the electrodes. On the other hand, the pressure has to be kept as low as possible for the user's comfort. This requirement implies specific limitations for the fixation arrangement making a design thereof a complicated task.

It is a purpose of the invention to provide a wearable system for enabling a bioelectrical interaction with an individual, in which the fixation of the electronic device to the carrier is simplified.

The wearable system according to the invention is characterized in that the electrodes are suitably shaped to enable a fixation of the electrodes on the carrier, said carrier being provided with receiving portions arranged to accommodate the electrodes. The technical measure of the invention is based on the insight that a body of the electrode can serve as the fixation arrangement, provided a proper shaping thereof. In the context of the present invention a wearable system is provided where the electronic device is self-fixating by means of the electrodes, simplifying the system design as a whole. Additionally, the wearable system is thus made versatile and can be mounted on any carrier adapted to receive the electrodes. An additional advantage of this set-up results in a simplicity of maintenance, especially when washing of the carrier is required. According to the invention the problem of washable connectors is eliminated, as due to the suggested system design all electronics is removed prior to washing or other maintenance operations. Therefore, the system according to the invention is easy to use and to maintain and this system can be arranged to carry out different bioelectrical operations.

An embodiment of the system according to the invention is characterized in that the electronic device further comprises electronics arranged to operate with the electrodes in order to enable said interaction, the electrodes and the electronics being integrated into one removable unit. This technical measure further simplifies the set-up of the wearable system. In view of presently achievable miniaturization of electronics, the electronics necessary to enable the envisaged application and the electrodes are integrated. The resulting unit can be made sufficiently compact to enable the fixation thereof on the carrier by means of the electrodes. Preferably, the electrodes are given the shape of buttons and the carrier is provided with cut-outs or slide-ins of a corresponding dimension. It is preferable to design the electrodes in the shape of a substantially semi-sphere, which will contribute to the immobilization of the electrodes on the skin of the individual when being worn. It is understood that forces applied to the electrode during a motion of the individual will result in a rotation of the electrode around its top, rather then in a displacement, resulting in an improvement of a signal to noise ratio of the system. Suitable materials for manufacturing the electrodes are conductive rubber are conductive plastic. These materials are preferable as they can be used as ‘dry’-electrodes without any gel for enabling an electrical contact.

A further embodiment of the system according to the invention is characterized in that the carrier is integrated into clothing. It is understood that for purposes of durable application it is advantageous to make the system as a part of clothing improving the wearing comfort and privacy for the user. For example, suitable clothing articles for integration with the wearable system according to the invention are sports brassier, boxer pants, or similar articles where a belt exhibiting substantial pressure on the electrodes is available.

A still further embodiment of the system according to the invention is characterized in that bioelectrical interaction comprises a monitoring of a vital sign by means of measuring an electrical signal on the individual's skin using the electrodes. It is particularly advantageous to use the system according to the invention for purposes of a durable monitoring of a vital sign. Especially, for cardiac applications, such as monitoring of a cardiac activity it is important to use a comfortable and easy to use wearable system. The principles of monitoring of a cardiac activity by means of a system with body electrodes is known in the art and will not be explained in detail here.

These and other aspect of the invention will be discussed with reference to the Figures.

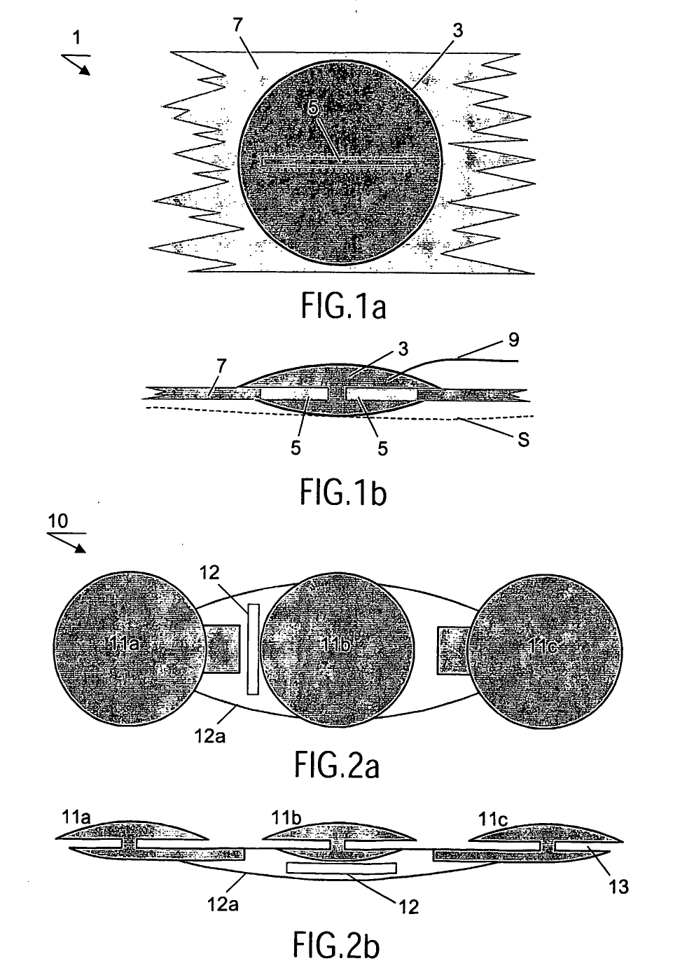

FIG. 1 shows a schematic view of an embodiment of a suitable electrode-carrier configuration to be used in the wearable system according to the invention.

FIG. 2 shows schematically an embodiment of an integrated electronic device to be used in the wearable system according to the invention.

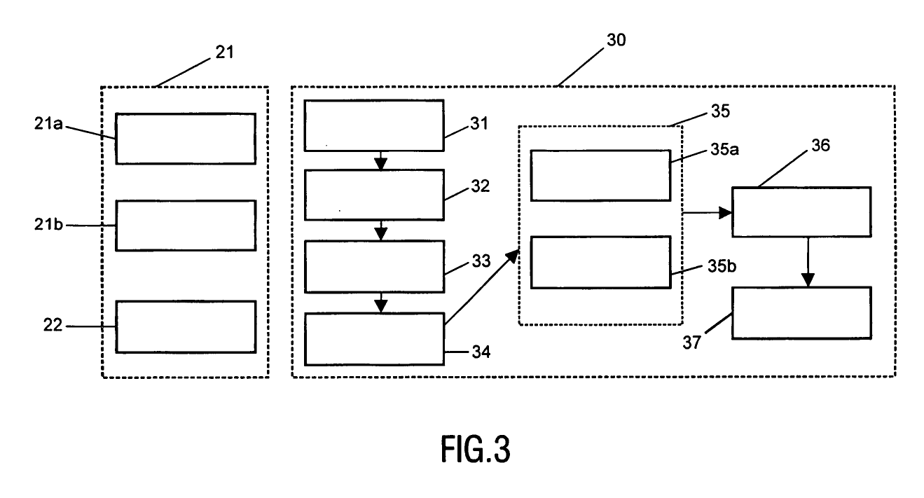

FIG. 3 shows an embodiment of a technical realization of a cardiac monitoring system according to the invention.

FIG. 1 presents a schematic view of an embodiment of a suitable electrode-carrier configuration 1 to be used in the wearable system according to the invention. A top view (a) of the electrode-carrier configuration 1 schematically illustrates a projection of a circular electrode 3 on a carrier 7. A side view (b) schematically illustrates a placement of the assembly 1 on the individual's skin S. In an example illustrated in FIG. 1, an elastic belt serves as an appropriate carrier 7 for the wearable monitoring system. A selection of an elastic belt as a carrier 7 is particularly suited for applications, like an electrostimulation of muscle activity, a monitoring of a physical activity during an exercise. Receiving portions 5 are provided in the body of the carrier 7 in order to accommodate the electrodes 3. The present example illustrates an embodiment of a receiving portion 5, being a cut-out in the material of the carrier 7. Alternatively receiving portions can be manufactured as pockets with a cut-away at places previewed for enabling contact between the material of the electrode 3 and the individual's skin S. It must be noted that the electrodes can be manufactured in different shapes, namely circular, oval, rectangular, etc. Preferably, a surface of the electrode facing the individual's skin is rounded off, which ensures a better immobilization of the electrode with respect to the individual's skin S. The preferred electrode type for this application is a dry-type electrode, the preferred electrode material is conductive rubber or conductive plastic. Both materials have sufficient rigidity to be used as a fixation arrangement and can be used to conduct electric signals measured on the individual's skin. Preferably, the electrodes are manufactures from a single material, however it is also possible to manufacture the surface facing the skin from the conductive rubber or conductive plastic and to select a different material, for example copper or other suitable metal for the remaining volume of the electrode. This material set-up can improve the conducting properties of the electrode. In order to establish an electrical connection with the electronics of the electronic device of the wearable system, leads 9 are provided to the electrodes 3. The functioning of the electronics is known per se in the art and will not be explained in detail.

FIG. 2 shows schematically an embodiment of an integrated electronic device 10 to be used in the wearable system according to the invention. FIG. 2a presents a top view of an integrated unit 10, and FIG. 2b presents a side view of the integrated unit 10. It is considered to be advantageous to integrate the electrodes 11a, 11b, 11c with a housing 12a of the electronic means 12 of the wearable system according to the invention. In the housing 12a electronic means 12 like an ECG amplifier or an electrostimulator can be accommodated. The placement of the electrodes 11a, 11b, 11c with respect to the housing 12a may vary. It is possible to code the orientation of the device by providing an asymmetric receiving portion on the belt 13 and by positioning the electrodes on the housing correspondingly. This set-up is particularly suited for durable applications, like monitoring of a vital sign, as there is no obstacle for movements of the user. For durable applications the carrier 13 is integrated into clothing, like sportswear or underwear which enables user friendliness and comfort and ensures privacy for the user.

FIG. 3 shows an embodiment of a technical realization of a cardiac monitoring system according to the invention. The monitoring system 20 comprises monitoring means 21 arranged to monitor a physiological condition of the user. The monitoring means 21 comprise a set of electrodes 21a, 21b to be arranged on the body of the user to pick-up a signal characteristic of the cardiac activity, for example an ECG signal. Additionally, the monitor means 21 can comprise a sensor 22 arranged to monitor a signal not directly related with a targeted physiological condition. An example of such a sensor is a motion sensor, or a blood pressure sensor. The monitoring means 21 are arranged to perform a continuous monitoring of the cardiac activity of the user and are further arranged to provide a corresponding signal to the front-end electronics 30 of the system 20. The monitoring means 21 and the front-end electronics 30 are worn on the body of the user, preferably at the waist area. An example of the preferred embodiment of a body worn system is shown in FIG. 2. The front-end electronics 30 is arranged to analyze said signal in order to derive a feature characteristic to an abnormality in the cardiac condition of the user. For that purpose the front-end electronics 30 comprise a preamplifier 31 and analogue processing circuit 32, an ADC unit 33, a μ-processor 34, detection means 35, alarm means 36 and transmission means 37. The detection means 35 comprise a sensor signal interpretation unit 35a and feature extraction means 35b. The system 20 operates as follows: the monitoring means 21 acquire the raw data which are delivered to the front-end electronics 30. The front-end electronics 30 provides means for receiving the signals from the monitoring means, performs suited analogue processing by means of the analogue processing circuit 32. The processed raw data is converted into a digital format by means of the ADC 33 and is forwarded by a μ-processor 34 to the detection means 35, where the condition of the user is being analyzed. For cardiac applications the detection means 35 comprise a per-se known QRS-detector to determine R-R peak intervals in heart cycles. The detection means 35 comprise a sensor signal interpretation unit 35a arranged to derive a feature in the signal characteristic to an abnormal physiological condition of the user. For cardiac said feature can be a frequency of the signal. In case the detection means 35 detects the abnormal condition, a signal is sent to the alarm means 36 to generate an alarm, which is transmitted by the transmitting means 37, for example by means of a RF-link to warn a bystander or specialized medical personnel.

1. A wearable system (1) arranged for enabling a bioelectrical interaction with an individual when being brought into contact with the individual's skin (S), said system comprising an electronic device arranged to be mounted on a carrier (7), said electronic device comprising electrodes (3) arranged to carry out said interaction, characterized in that said electrodes (3) are suitably shaped to enable a fixation of the electrodes on the carrier (7), said carrier being provided with receiving portions (5) arranged to accommodate the electrodes (3).

2. A system according to claim 1, characterized in that the electronic device further comprises electronic means (12) arranged to operate with the electrodes (11a, 11b, 11c) in order to enable said interaction, the electrodes and the electronic means being integrated into one unit arranged to be removably attached to the carrier.

3. A system according to claim 1 or 2, characterized in that a material of the electrodes comprises conductive rubber.

4. A system according to claim 1 or 2, characterized in that a material of the electrodes comprises plastic.

5. A system according to any one of the preceding claims, characterized in that the electrodes (3) are button-shaped, the receiving portions of the carrier are notches (5) of suitable dimension.

6. A system according to any one of the preceding claims, characterized in that the carrier (7) is integrated into clothing.

7. A system according to any one of the preceding claims, characterized in that the bioelectrical interaction comprises a monitoring of a vital sign by means of measuring an electrical signal on the individual's skin using the electrodes.

8. A system according to claim 7, characterized in that the vital sign is a cardiac activity of the individual.
